(19) United States
(12) Patent Application Publication
(10) Pub. No.: US 2016/0001262 A1
(43) Pub. Date: Jan. 7, 2016

(54) MIXED MULTIFUNCTIONAL METAL AFFINITY SURFACES FOR REDUCING AGGREGATE CONTENT IN PROTEIN PREPARATIONS

(71) Applicant: AGENCY FOR SCIENCE, TECHNOLOGY AND RESEARCH, Connexis (SG)

(72) Inventor: Peter Stanley GAGNON, Centros (SG)

(21) Appl. No.: 14/766,126

(22) PCT Filed: Feb. 5, 2014
(86) PCT No.: PCT/SG2014/000045

§ 371 Date: Aug. 5, 2015

Related U.S. Application Data
(60) Provisional application No. 61/761,653, filed on Feb. 6, 2013.

(51) Int. Cl. B01J 20/24; B01J 20/288; B01D 15/38; C07K 1/16

(52) U.S. Cl. CPC B01J 20/24; C07K 1/165; B01J 20/288; B01D 15/3828; B01J 2220/80

(57) ABSTRACT

A composition for reducing the aggregate content of a protein preparation includes a first substrate having a first surface-bound ligand possessing a metal affinity functionality, the metal affmity functionality being substantially devoid of a metal, and an additional surface-bound ligand different from the first surface-bound ligand, the additional surface-bound ligand having an aggregate charge not opposite to that of the metal affinity functionality, optionally the additional surface-bound ligand is provided on an additional substrate such that the composition comprises a mixture of the first substrate and the additional substrate.

### STATEMENT OF RELATED APPLICATIONS

This application claims priority U.S. 61/761,653, filed Feb. 6, 2013, the entirety of which is incorporated herein by reference.

### BACKGROUND

This invention relates to materials for purification of proteins, especially antibodies. It particularly relates to materials for reducing the content of aggregates, and especially aggregates that include host cell chromatin remnants such as nucleosomes, histones, and DNA.

It has been indicated that unnatural hetero-aggregates form spontaneously between host cell-derived contaminants and recombinant proteins produced by in vitro cell culture methods (Shukla et al., Biotechnol. Progr. (2008) 24:1115-1121; Luhrs et al., J. Chromatogr. B (2009) 877:1543-1552; Mechetner et al., J. Chromatogr. B (2011) 879:2583-2594; Gagnon et al., J. Chromatogr. A, (2011) 1218:2405-2412; Gagnon, Bioprocessing J. (2010) 9(4):14-24). These hetero-aggregates may be considered unnatural in two respects: 1) constituent contaminants are often of non-human origin, secreted by living non-human host cells or released into the culture media when non-human host cells lyse upon death. In living humans, such non-human contaminants do not exist; and 2) constituent contaminants accumulate to high concentrations in comparison to human in vivo systems where dead cell constituents are quickly eliminated. Accordingly, recombinant products are exposed to high levels of strongly interactive contaminants at concentrations that typically do not occur in living systems. Meanwhile, high expression levels of recombinant proteins make them suitable substrates for non-specific associations with these non-human contaminants, favoring the formation of undesirable hetero-aggregates of diverse composition.

The contaminating protein content of hetero-aggregates has been addressed to some extent via direct targeting of the contaminating protein (Shukla et al. and Gagnon et al. supra), as well as indirectly via targeting of the corresponding DNA component responsible for the contaminating protein (Luhrs et al. and Gagnon supra). A reduction of antibody aggregate level has been indicated when some complexes are dissociated (Shukla et al., Mechetner et al., and Gagnon supra). The ability of anion exchangers to reduce levels of antibody-contaminant complexes has been disclosed (Luhrs et al. and Gagnon et al. supra), but an anion exchange treatment that was able to fully eliminate hetero-aggregates has not been indicated. Size exclusion, cation exchange, and hydrophobic interaction chromatography have also been employed in attempts to reduce hetero-aggregates, but these techniques were generally inferior to anion exchange (Gagnon et al. supra).

The specific source of contaminants that form stable associations with antibodies is not always known (see, for example, Shukla et al. supra). Some efforts have focused on DNA contaminants with little attention to the specific source of other possible contaminants (Gagnon et al. and Gagnon supra). Some efforts indicating an association of host contaminants with aggregates in antibody preparation have focused specifically on contaminants comprising chromatin catabolites (Luhrs et al. and Mechetner et al. supra). In these examples, aggregation may be mediated directly through the immunospecificity of the antibody for chromatin catabolites such as histones and DNA. It has been indicated that chromatin catabolites are also capable of forming stable complexes with antibodies via non-specific interactions. Thus, monoclonal antibodies with known immunospecificities for antigens not including chromatin catabolites, can form highly stable aggregates of diverse descriptions with nucleosomes, histones, and DNA derived from the nuclei of dead host cells (Gan et al, J. Chromatography A 191 (2013) 33-40). It has been particularly indicated that chromatin catabolites are highly represented in high molecular weight (HMW) aggregates. HMW aggregates are of particular concern because of their suspected involvement in promoting the formation of therapy-neutralizing antibodies. HMW aggregates are generally defined as aggregates of a size greater than small multiples of the antibody of interest. For example, 2-antibody associations are not considered HMW aggregates, nor are most 4-antibody aggregates. However, aggregates of much greater size, such as corresponding to about 8 to about 10 or more antibodies may be generally classified as HMW aggregates.

Treating antibody preparations with agents that might be expected to dissociate hetero-aggregates has generally proven ineffective. For example, employing high concentrations of urea, salts, or combinations of the two does not substantially dissociate IgM-contaminant hetero-aggregates (Gagnon et al. supra). Protein A affinity chromatography with pre-elution washes of urea, alcohol, and surfactants has been indicated to reduce hetero-aggregate levels more effectively than without washes (Shukla et al. supra), as did pre-elution washes combining urea, salt, and EDTA with protein G affinity chromatography (Mechetner et al. supra). Anion exchange chromatography with a pre-elution wash of urea has been indicated to reduce hetero-aggregates-more effectively than in the absence of a urea wash (Gagnon et al. supra). Cation exchange chromatography has also been indicated to reduce hetero-aggregates more effectively with a pre-elution EDTA wash than without the wash (Gagnon et al. supra). Finally, hydroxyapatite with pre-elution washes of urea and/or salt have also reduced hetero-aggregates more effectively than without such washes (Gagnon supra). Despite these observations, in general, the use of dissociating agents in pre-elution washes of antibodies bound to chromatography columns has been only moderately successful.

Immobilized TREN (tris(2-aminoethyl)amine) is known and commercially available for the purpose of conducting immobilized metal affinity chromatography (IMAC), where the immobilized TREN is initially loaded with a metal ion, and biomolecules are captured by contact with the TREN-associated metal ion, then subsequently recovered by dissociating the target biomolecule from the metal ion. IMAC ligands other than TREN, such as iminodiacetic acid (IDA) and nitriloacetic acid (NTA), are also known and commercially available for the purpose of conducting IMAC, where the ligand is initially loaded with a metal ion, and biomolecules are captured by contact with the ligand-associated metal ion, then subsequently recovered by dissociating the target biomolecule from the metal ion.

### SUMMARY

In some aspects, embodiments disclosed herein relate to compositions for reducing the aggregate content of a protein preparation, the composition comprising a first substrate comprising a first surface-bound ligand possessing a metal affinity functionality, the metal affinity functionality being substantially devoid of a metal (metal-ion), and an additional surface-bound ligand different from the first surface-bound ligand, the additional surface-bound ligand having an aggregate charge not opposite to that of the metal affinity functionality, wherein optionally the additional surface-bound ligand is provided on an additional substrate whereby the composition comprises a mixture of the first substrate and the additional substrate.

### DETAILED DESCRIPTION

It has been surprisingly discovered that compositions disclosed herein comprising solid materials bearing a metal affinity ligand with a negative net charge and at least one additional ligand bearing a negative net charge or no net charge, or alternatively comprising solid materials bearing a metal affinity ligand with a positive net charge and at least one additional ligand bearing a positive net charge or no net charge, has the ability to substantially reduce the content of high molecular weight (HMW) aggregates and aggregates of smaller size in protein preparations. Certain embodiments of the invention have the additional ability to remove agents such as multivalent ions and antiviral compounds that may have been added to the protein preparation.

In certain embodiments, the invention provides devices where the respective ligands not of opposite charge may reside on the same surface, or on different surfaces, or a mixture of both. Such ligands may possess or be combined with additional chemical functionalities including but not limited to hydrophobic, pi-pi bonding, hydrogen bonding, and metal affinity. The solid surfaces may be particulate, fibrous, porous-membranaceous, or monolithic in structure, including combinations of multiple structural types. In some embodiments, the respective ligands not of opposite charge may reside on one, two, three, four or more separate surfaces, including any of the aforementioned structural types. Where multiple surfaces are employed, one, two, three, four, or more functionalities may be provided by any number of ligands for each surface. A single ligand may also provide one, two, three, four, or more functionalities. By way of example, a single ligand may provide two functionalities such as metal affinity as well as hydrogen bonding functionality, or metal affinity and hydrophobic functionality, or metal affinity and pi-pi bonding functionality, and so on. Likewise, a single ligand may also provide three functionalities such as metal affinity, hydrophobic functionality and pi-pi bonding functionality, and so on. Those skilled in the art will recognize the ability to combine any desired types and numbers of functionalities and distribute them among any number of ligands and any number of surfaces bearing those ligands.

In certain embodiments, the device may be configured in such a way that contact of an applied sample with the respective ligands not of opposite charge is substantially simultaneous. In some such embodiments, where multiple surfaces are being employed, they may be mixed homogeneously or they may appear in a gradient mixture.

In other embodiments, the device may be configured in such a way that contact of an applied sample with the respective ligands not of opposite charge is sequential. In some such embodiments, sequential contact may be facilitated by the use of multiple surfaces and such multiple surfaces may be spatially separated. In other such embodiments, sequential contact may be facilitated by contacting the sample first with one surface, then later with another. In other such embodiments, sequential contact may be facilitated by both spatial and temporal separation.

The invention provides in certain embodiments, compositions of matter and apparatus for the purification of proteins, including the reduction of aggregate concentration in antibody preparations. In certain embodiments, a composition of matter for reducing the aggregate content of a protein preparation is provided where the composition includes a first surface-bound ligand possessing metal affinity functionality and at least one additional surface-bound ligand having an aggregate charge not opposite to that of the metal affinity functionality of the first surface-bound ligand.

In certain embodiments, the first surface-bound ligand and an additional surface-bound ligand are each covalently bound to a substrate. The first surface-bound ligand and the additional surface-bound ligand may be each covalently bound to the same substrate. In other embodiments, the first surface-bound ligand and the additional surface-bound ligand are each covalently bound to different substrates. The substrate may be a particle and in certain embodiments the particle may be porous or non-porous. In certain embodiments, the substrate is a porous particle having pores large enough to permit entry of a protein in a protein preparation. In other embodiments, the pores are too small to permit entry of a protein in a protein preparation. In certain embodiments the substrate is porous particle having an average pore size between about 10 nm and about 100 nm, less than about 10 nm, or above about 100 nm. In certain embodiments, the substrate is a membrane, a monolith, or a solid or porous walled hollow fiber. In certain embodiments the substrate to which the first surface-bound ligand is attached is of a different kind than the substrate to which an additional surface-bound ligand is attached. For example one substrate may be a particle while the other substrate may be a monolith, membrane, or fiber, porous-walled hollow fiber, plurality or compound construct of the foregoing.

In certain embodiments, the first surface-bound ligand and an additional surface-bound ligand are different. In certain embodiments, the first surface-bound ligand is a multidentate metal chelating moiety. In certain embodiments, the first surface-bound ligand has an aggregate charge which is electronegative. In others, the first surface-bound ligand has an aggregate charge which is electropositive. In certain embodiments, an additional surface-bound ligand possesses metal affinity functionality. In certain such embodiments, the additional surface-bound ligand is a multidentate metal chelating moiety.

In certain embodiments, the first surface-bound ligand is electropositive and that surface has additional chemical moieties bound to it, provided that the aggregate charge of that surface is electropositive. In some such embodiments, one or more of the additional chemical moieties may have an opposite (negative) charge so long as the aggregate charge remains positive. In other such embodiments, the first surface-bound ligand is electronegative and that surface has additional chemical moieties bound to it, provided that the aggregate charge of that surface is electronegative. In some such embodiments, one or more of the additional chemical moieties may have an opposite (positive) charge so long as the aggregate charge remains negative. In certain embodiments, at least one of the substrates has one or more chemical moieties in addition to the first surface-bound ligand or an additional surface-bound ligand wherein such additional chemical moieties enhance the capacity of the composition to participate in hydrogen bonding, hydrophobic interactions, metal affinity or pi-pi binding with a protein of the protein preparation.

In certain embodiments, the first surface-bound ligand is electronegative and is iminodiacetic acid (2-(carboxymethylamino)acetic acid) (EDTA), ethylene glycol(aminoethylether) diacetic acid (EGTA), nitriloacetic acid (2,2′,2″-Nitrilotriacetic acid) (NTA), aspartic acid, or glutamic acid. In certain embodiments, the first surface-bound ligand is electropositive and is tris(2-aminoethyl)amine or desferioxamine.

In certain embodiments, the surface-bound chemical moieties described in the foregoing embodiments may alternatively be directly incorporated in the structure of the polymer or polymers during synthesis of the physical surface.

In certain embodiments, the invention provides an apparatus configured for chromatography including a composition of the invention. In certain embodiments, the apparatus consists of a housing that contains the substrate or substrates to which the first surface-bound ligand and an additional surface-bound ligand are attached. In certain embodiments, the apparatus contains one or more porous membranes and at least one of such membranes is the substrate to which the first surface-bound ligand and an additional surface-bound ligand are attached. In certain embodiments, the membranes are in the form of porous-walled hollow fibers. In certain embodiments, the apparatus contains a porous reticular arrangement of fibers, where such fibers are the substrates to which the first surface-bound ligand and an additional surface-bound ligand are attached.

In certain embodiments, the apparatus contains porous or non-porous particles sandwiched between porous membranes, or monoliths, or frits. In certain such embodiments, the particles are the substrate to which either the first surface-bound ligand or an additional surface-bound ligand is attached. In certain embodiments, the first surface-bound ligand and an additional surface-bound ligand may also be attached to the membrane or monolith. In certain embodiments, the apparatus contains porous or non-porous particles sandwiched between woven or amorphous fibrous filters. In certain such embodiments, the particles are the substrate to which either the first surface-bound ligand or an additional surface-bound ligand is attached, and the fibrous filters are substantially inert. In other embodiments, the fibrous filters may also bear one or more surface-bound ligands. In certain embodiments, the apparatus contains porous or non-porous particles sandwiched between woven or crystalline frits, and the frit is substantially inert. In certain embodiments, the frits may bear one or more surface-bound ligands. In certain embodiments, the apparatus contains porous or non-porous particles embedded in a reticular polymer network. In certain such embodiments, particles are the substrate to which either the first surface-bound ligand or the second surface-bound ligand is bound and the reticular polymer network is substantially inert. In other embodiments the reticular polymer network also bears one or more surface-bound ligands. In certain embodiments, the apparatus provides where both the substrate to which the first surface-bound ligand is bound and the substrate to which an additional surface-bound ligand is bound are both particles and the particles are confined between membranes, monoliths, a reticular polymer network, woven or amorphous fiber filters, crystalline frits, or a combination thereof.

In certain embodiments, the chemical surface of one or more components of the apparatus may be relatively inert or of such a relatively low surface area as to make no significant contribution to the chemical functionality of the apparatus. In certain such embodiments, the chemical surface which is relatively inert or of relatively low surface area is configured so as to create structural integrity, or direct flow of liquids therethrough, or physically block, entrap, or entrain insoluble materials in a protein preparation to prevent them from interfering with the effective use of the device.

In some embodiments, a ratio of the first surface-bound ligand and an additional ligand may be in a range of from about 1:99 to about 99:1. Those skilled in the art will appreciate that almost any ratio may be appropriate for a particular application. As a matter of practicality, in some embodiments, optimizing a ratio of first surface to second surface may begin by employing an equitable 1:1 ratio of the first and second components and optimizing the ratio systematically by altering the ratios from this starting point.

In some embodiments where the ligands reside on materials of multiple types, the ratio of and distribution of the respective materials may be of diverse character. Those skilled in the art will appreciate that almost any ratio and/or distribution may be appropriate for a particular application.

Terms are defined so that the invention may be understood more readily. Additional definitions are set forth throughout the detailed description.

“Aggregate(s)” refers to an association of two or more molecules that is stable at physiological conditions and may remain stable over a wide range of pH and conductivity conditions. Aggregates frequently comprise at least one biomolecule such as a protein, nucleic acid, or lipid and another molecule or metal ion. The association may occur through any type or any combination of chemical interactions. Aggregates of antibodies can be classified into two categories: “Homoaggregates” refers to a stable association of two or more antibody molecules; “Hetero-aggregates” refers to a stable association of one or more antibody molecules with one or more non-antibody molecules. The non-antibody component may consist of one more entities from the group consisting of a nucleotide, an endotoxin, a metal ion, a protein, a lipid, or a cell culture media component.

“Antibody” refers to an immunoglobulin, composite, or fragmentary form thereof. The term may include but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” may also, include composite forms including but not limited to fusion proteins containing an immunoglobulin moiety. “Antibody” may also include antibody fragments such as Fab, F(ab′)₂, Fv, scFv, Fd, dAb, Fc and other compositions, whether or not they retain antigen-binding function.

“Endotoxin” refers to a toxic heat-stable lipopolysaccharide substance present in the outer membrane of gram-negative bacteria that is released from the cell upon lysis. Endotoxins can be generally acidic due to their high content of phosphate and carboxyl residues, and can be highly hydrophobic due to the fatty acid content of the lipid-A region. Endotoxins can offer extensive opportunity for hydrogen bonding.

“Substrate” or “Solid material” refers to an insoluble organic or inorganic solid that may be particulate, crystalline, polymeric, fibrous, porous-hollow fibrous, monolithic, or membranaceous in nature. It may consist of non-porous or porous particles, a porous membrane, a porous filter, or a porous monolith. If particulate, the particles may be roughly spherical or not, and may be of sizes ranging from less than 100 nm to more than 100 microns. The average pore size of porous particles may range less than 10 nm (microporous) to more than 100 nm (macroporous). The average pore size in membranes may range from less than 100 nm to more than 1 micron. The average channel size in membranes or monoliths may range from less than 1 micron to more than 10 microns. The solid material may further consist of compound constructions, for example in which particles are embedded in a reticular matrix, sandwiched between membranes, or both.

“Metal affinity functionality” refers to the capacity of a chemical moiety, which may be immobilized on a surface, to bind metal ions preferably in a 1:1 fashion. Such moieties may have the capacity to form coordination bonds with a metal ion and certain such moieties may be bidentate or multidentate in character. Nonlimiting examples of electronegative moieties with this capability include iminodiacetic acid (2-(carboxymethylamino)acetic acid), diethylamine triamine pentacetic acid, and nitriloacetic acid (2,2′,2″-nitrilotriacetic acid). Examples of electropositive compounds with this capability include but are not limited to Tris(2-aminoethyl)amine, diethylenetriamine, triethylenetetramine, tetraethylenepentamine, polypropylenimine tetraamine, and desferrioxamine.

“Electropositive surface” refers to a surface of a substrate or solid material which is dominated by positive charge. Electropositivity of a surface may be conferred by chemical groups including but not limited to weak anion exchange groups, like amino, ethylene diamino, diethylaminoethyl, polyallylamine, polyethyleneimine, strong anion exchange groups, such as quaternary amino groups, combined weak-strong exchangers, such as polylysine, polyarginine, or Tris(2-aminoethyl)amine, diethylenetriamine, triethylenetetramine, tetraethylenepentamine, polypropylenimine tetraamine, PAMAM dendrimer (ethylenediamine core), or any combinations of the foregoing. Secondary functionalities that create a mixed chemical character on a positively charged surface may consist of negatively or positively charged groups, hydrophobic groups, pi-pi bonding groups, hydrogen-bonding groups, or metal-chelation groups. The secondary functionalities may exist on electropositive surfaces as an inadvertent byproduct of the manufacturing materials or process by which the particles are synthesized, or they may be present by deliberate design. The concentration of secondary functionalities may range from less than 1 milliequivalent per mL of particles, to more than 100 milliequivalents per Ml.

“Electronegative surface” refers to a surface of a substrate or solid material which is dominated by negative charge. Electronegativity of a surface may be conferred by chemical groups including but not limited to so called weak cation exchangers, such as carboxyl, aminocarboxyl (iminodiacetic or nitriloacetic), or phosphoryl, or strong exchangers such as sulfo groups (e.g., sulfo, sulfomethyl, sulfoethyl, sulfopropyl). Secondary functionalities that create a mixed chemical character on a negatively charged surface may consist of negatively or positively charged groups, hydrophobic groups, pi-pi bonding groups, hydrogen-bonding groups, or metal-chelation groups. The secondary functionalities may exist on electronegative surfaces as an inadvertent byproduct of the manufacturing process by which the particles are synthesized, or they may be present by deliberate design. The concentration of secondary functionalities may range from less than 1 milliequivalent per mL of particles, to more than 100 milliequivalents per mL.

“Polynucleotide” refers to a biopolymer composed of multiple nucleotide monomers covalently bonded in a chain. DNA (deoxyribonucleic acid) and RNA (ribonucleic acid) are examples of polynucleotides. Polynucleotides can have a high propensity for formation of hydrogen bonds.

“Protein” refers to any of a group of complex organic macromolecules that contain carbon, hydrogen, oxygen, nitrogen, and usually sulfur and are composed principally of one or more chains of amino acids linked by peptide bounds. The protein may be of natural or recombinant origin. Proteins may be modified with non-amino acid moieties such as through glycosylation, pegylation, or conjugation with other chemical moieties. Examples of proteins include but are not limited to antibodies, clotting factors, enzymes, and peptide hormones.

“Protein preparation” refers to any aqueous or mostly aqueous solution containing a protein of interest, such as a cell-containing cell culture harvest, a (substantially) cell-free cell culture supernatant, or a solution containing the protein of interest from a stage of purification.

“Virus” or “virion” refers to an ultramicroscopic (roughly 20 to 300 nm in diameter), metabolically inert, infectious agent that replicates only within the cells of living hosts, mainly bacteria, plants, and animals: composed of an RNA or DNA core, a protein coat, and, in more complex types, a surrounding envelope.

In certain embodiments, the solid materials used to practice the invention may include insoluble particles of natural or synthetic origin, such as but not limited to porous microparticles commonly employed for practicing chromatography. Such particles may embody large pores that permit the diffusive entry of proteins, such as but not limited to antibodies; or they may embody small pores that allow the diffusive entry of small chemical species such as salts, sugars, and hetero-aggregate-dissociating agents, but are too small to permit the entry of proteins such as antibodies. The solid materials may alternatively include non-porous particles, membranes or monoliths, fibers including porous-walled hollow fibers, porous membranes, and/or compound constructions employing combinations of the above elements.

Electropositive groups may include so-called strong anion exchange groups and/or so-called weak anion exchange groups. The term strong anion exchanger includes functional groups such as quaternary amines, which embody pKas above pH 12. The term weak anion exchanger is understood to refer to functional groups such as diethylaminoethyl, and ethylenediamine, which embody pKas lower than 12. Electropositive groups of weak or mixed strong-weak anion exchange may be included. A non-limiting example of such a mixed weak-strong anion exchange group with metal coordination ability is Tris(2-aminoethyl)amine (TREN), which immobilized on a surface may contain primary, secondary, and tertiary amino groups. In some such embodiments, TREN already immobilized on a surface may undergo secondary chemical modification with the objective of attaching an additional layer of TREN groups to the already-immobilized TREN groups, thereby creating a TREN dendrimer that extends further from the surface. In some such embodiments, another layer of TREN groups may be added to the second layer of TREN groups, creating a 3-layer TREN dendrimer, and so on.

Electronegative groups may include so-called strong cation exchange groups or so-called weak cation exchange groups. The term strong cation exchanger is understood to include sulfate or sulfo containing moieties with pKas below 3. The term weak cation exchanger is understood to include moieties containing carboxy and/or phospho groups with pKas above 3. Dominantly electronegative groups of dipolar character (at neutral pH), such as combinations of two carboxyl groups with an amino group may be included. Non-limiting examples of such groups include iminodiacetic (IDA), ethylene glycol(aminoethylether) diacetic acid (EGTA), and nitriloacetic acid (NTA) groups, among others.

The surfaces of the electropositive or electronegative materials may also incorporate hydrophobic groups of an aliphatic and/or or aromatic character, where the latter may be preferred because of their ability to participate in so-called pi-pi binding. Mixed chemical character may reside in a single complex chemical group, in separate chemical groups of distinct character on a single type of surface, on distinct surfaces, or any combination of the foregoing. One or more electronegative metal affinity and/or one or more electropositive metal affinity groups may be employed simultaneously, and they may differ with respect to the physical form in which they are embodied. Chemical functionalities of differing individual character may be employed in various ratios customized to the needs of a particular sample composition. For example, combinations intended for treatment of clarified cell culture supernatant may include an excess of electropositive surfaces, while combinations intended for treatment of supernatant already treated with soluble agents such as ethacridine, methylene blue, cetyltrimethylammonium, chlorhexidine, or polyethyleneimine may include an excess of electronegative surfaces.

In certain embodiments, the invention may provide particles with an electronegative metal affinity functionality, combined with electronegative and/or electro-neutral particles which may be mixed together and which may further be mixed with and/or enclosed by neutral materials, embedded in neutral materials, or enclosed by or embedded in materials that are themselves electronegative.

In certain embodiments, the invention may provide particles with an electropositive metal affinity functionality, combined with electropositive and/or electroneutral particles which may be mixed together and which may further be mixed with and/or enclosed by neutral materials, embedded in neutral materials, or enclosed by or embedded in materials that are themselves electropositive.

In certain embodiments, the invention may provide particles with an electronegative metal affinity functionality, combined with particles with an electronegative or electro-neutral metal affinity functionality which may be mixed together and which may further be mixed with and/or enclosed by neutral materials, embedded in neutral materials, or enclosed by or embedded in materials that are themselves electronegative and/or electropositive.

In certain embodiments, the invention may provide electronegative and/or electro-neutral surfaces which may embody additional chemical functionalities, including but not limited to the ability to participate in hydrophobic interactions, pi-pi bonding, hydrogen bonding, and metal affinity. In certain embodiments, the invention may provide one or more types of electronegative particles and one or more types of electroneutral particles mixed together and enclosed by neutral materials.

In certain embodiments, the particles may have differing sizes and/or differing porosities.

In certain embodiments, the invention may provide particles which may be mixed with and/or enclosed between not-oppositely-charged materials of other physical form, including but not limited to membranes, fibers, and monoliths.

In certain embodiments, differing proportions of first and additional functionalities on one or more substrates may be selected to accommodate the needs of protein preparations of differing composition.

It will be apparent to the person of ordinary skill in the art that, in addition to being useful for reducing the content of homoaggregates and hetero-aggregates in a protein preparation, that in certain embodiments, the invention may be useful for substantially reducing the content of host cell protein, polynucleotides, endotoxin, and virus from a protein preparation. In the case that the primary functionalities are accompanied by additional functionalities, such as hydrophobic and hydrogen bonding for example, the invention may also reduce content of cell culture media components and additives that limit the ability of downstream purification methods to reproducibly achieve their goals. It will also be apparent to the person of ordinary skill, that although certain embodiments of the invention may have substantial value when applied to relatively crude feed streams, it may nevertheless offer, in certain embodiments, important value when to applied samples that are substantially purified.

In certain embodiments, the invention may provide compositions or apparatus such that the solid materials may be cleaned and recycled after use. In other embodiments, they may be configured for single use.

Additional objects and advantages of the invention will be set forth in part in the description which follows, and in part will be obvious from the description, or may be learned by practice of the invention. The objects and advantages of the invention will be realized and attained by means of the elements and combinations specified in the claims.

It is to be understood that both the foregoing general description and the following detailed description are exemplary and explanatory only and are not restrictive of the invention, as claimed.

### EXAMPLES

### Example 1

1 L of cell culture harvest containing about 1 g/L of an IgG monoclonal antibody specific for HER2 antigen was treated with 1% allantoin. Conductivity was about 13 mS/cm and pH was about 6.8. The additives had the effect of accelerating sedimentation. Solid materials were removed by filtration, leaving a clear antibody-containing filtrate. Antibody recovery was 99%. 20 mL of BioWorks TREN hi-sub, an agarose porous particle-based electropositive metal affinity material was packed in a column (1.6×10 cm) and equilibrated to 50 mM Hepes, 150 mM NaCl, pH 7.0. The clarified filtrate was passed through the column at a linear flow rate of 200 cm/hr. The original harvest contained more than 20% aggregates, particularly containing at least 10% so-called high molecular weight aggregates. The treated sample contained less than 0.05% high molecular weight aggregates and less than 4% total aggregates. DNA as measure by fluorescent dye assay was reduced by greater than 98%, but qPCR indicated that reduction was actually greater than 99.999%. Histone proteins were reduced by at least 98% and general host protein levels, as measured by Cygnus ELISA was reduced by 62%. Antibody recover was 99%.

### Example 2

The procedure of Example 1 was repeated except substituting TREN for a 1:1 mixture of TREN plus Dowex AG1x2, a hydrophobic electropositive particulate material. All results were nominally the same, except that antibody recovery was reduced to 95% and analytical size exclusion chromatography showed that two strongly hydrophobic contaminants evident after Example 1, were eliminated.

### Example 3

The procedure of Example 2 was repeated except that cell-containing harvest was treated with 0.05% octanoic acid in addition to 1% allantoin. Host protein contamination was reduced by more than 70%, and antibody recovery was reduced to 90%. Octanoic acid was undetectable in the treated sample, appearing to indicate that it was bound to the solid material(s). Aggregates were reduced to less than 3%. DNA and histone content were reduced by 99%.

### Example 4

The procedure of Example 2 was repeated with an IgM monoclonal antibody, except that NaCl was added to the cell-containing harvest to a concentration giving a conductivity of 20 mS/cm and the pH was adjusted to 6.0. The aggregate content of the untreated harvest was greater than 30%. The column was equilibrated to 50 mM MES, 200 mM NaCl, pH 6.0. 99% of the DNA and histones were removed, along with all high molecular weight aggregates. Total aggregate content was reduced to about 2%. Antibody recovery was only 84%. Total host protein was reduced 67%. The experiment was repeated at a conductivity throughout of 25 mS/cm, corresponding to the addition of a 50 mM increment of sodium chloride over and above the concentration added to achieve a conductivity of 20 mS/cm. Antibody recovery increased to 98% and all other measures remained the same. The experiment was repeated at a conductivity of 40 mS/cm. Host protein reduction diminished to about 47%, but all other performance measures remained unchanged.

### Example 5

The procedure of Example 3 was repeated except substituting 0.025% ethacridine for octanoic acid, and using a column packed with acrylate-based porous particles bearing the metal affinity ligand iminodiacetic acid (Profinity, Bio-Rad) and styrene divinylbenzene particles (Chelex 100, Bio-Rad) in a 1:1 mixture. The treated sample was free of yellow color, while the chromatography media was intensely yellow, indicating removal of the ethacridine. DNA, histones, and nucleosomes were reduced by 99%, while antibody recovery was 99%. High molecular weight aggregates were completely eliminated, while total aggregates were reduced to less than 2%. Analytical SEC also demonstrated the removal of strongly hydrophobic contaminants. Total host protein contamination was reduced 63%.

### Example 6

The procedure of Example 5 was repeated except including negatively charged hydrophobic interaction particles (Macroprep T-butyl, Bio-Rad) mixed in a 1:1:1 ratio with the Profinity and Chelex. The treated sample was free of yellow color, while the chromatography media was intensely yellow, indicating removal of the ethacridine. DNA, histones, and nucleosomes were reduced by 99%, while antibody recovery was 99%. High molecular weight aggregates were completely eliminated, while total aggregates were reduced to less than 2%. Analytical SEC also demonstrated the removal of strongly hydrophobic contaminants. Total host protein contamination was reduced 64%.

### Example 7

Dynamic binding experiments were conducted on immobilized protein A (rAF protein A Toyopearl 650M, Tosoh), comparing the harvest clarified by centrifugation and microfiltration versus the treatment of Example 2. Dynamic binding capacity on the microfiltered material was 28 mg/mL. Dynamic binding capacity on the material from Example 3 was 35 mg/mL. Host protein content of the microfiltered material after protein A purification was 792 parts per million. Host protein content of the Example 3-treated material was less than 1 part per million.

### Example 8

The procedure of Example 2 was reproduced except increasing the operating pH to 8.0 and reducing the conductivity of the cell culture supernatant to 4.7 by dilution of the sample with 2 parts water and reformulating the buffer to contain 50 mM Tris, 50 mM NaCl, pH 8.0. General host protein reduction increased to 83% but aggregate removal was about 50% less effective than in Example 2.

### Example 9

The procedure of Example 2 was reproduced except increasing the overall proportion of particles from 2% to 5%. Results were nominally unchanged except that antibody recovery was reduced to 84%. In subsequent experiments where the proportion of total particles was 2%, and the proportion represented by Dowex AG1x2 was reduced to 25%, and 12.5% respectively, antibody recovery increased to 97 and 98% respectively. Removal of the hydrophobic contaminants was still effective at the reduced Dowex levels. All other results were nominally equivalent to Example 2.

### Example 10

The procedure of example 2 was repeated except replacing Dowex AG1x2 with UNOsphere Q. UNOsphere Q more effectively removed residual ethacridine. Results were otherwise similar.

### Example 11

The procedure of Example 3 was repeated except increasing caprylic acid to 0.4%, reducing the operating pH to 5.2, and incubating for 2 hours before addition of TREN particles, then incubating for 4 hours before removing the solids. Aggregate content was reduced to less than 0.1%. Host protein was reduced 99.9%.

### Example 12

The materials and procedure of Example 12, except for an operating pH of 5.6, were applied to a cell, culture harvest containing and IgM monoclonal antibody. Aggregates were reduced from an original 22% compared to the non-aggregated antibody, to less than 0.1%. Host proteins were reduced by more than 98%.

It will be apparent to the person of skill that the above examples employ chemical groups on particles, monoliths, membranes, and fibers; and in combinations of positively charged chelators with negatively charged groups, positively charged chelators with negatively charged chelators, positively charged groups with negatively charged chelators, and both combined with other chemical reactivities such as hydrophobic interaction ligands. Despite the diversity of these examples with IgG and IgM antibodies, it will be further apparent that many more such combinations could be conceived which, though different, manifest the essential features of the invention, and that it may be applied to many types of proteins other than antibodies where it can be expected to produce benefits similar to those illustrated above.

The present invention may be combined with various purification methods to achieve the desired levels of purification. Examples include, but are not limited to, other methods commonly used for purification of antibodies, such as protein A and other forms of affmity chromatography, anion exchange chromatography, cation exchange chromatography, hydrophobic interaction chromatography, immobilized metal affinity chromatography, and additional mixed mode chromatography methods. It is within the purview of a person of ordinary skill in the art to develop appropriate conditions for the various methods and integrate them with the invention herein to achieve the necessary purification of a particular antibody.

All references cited herein are incorporated by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

All numbers expressing quantities of ingredients, chromatography conditions, and so forth used in the specification and claims are to be understood as being modified in all instances by the term “about.” Accordingly, unless indicated to the contrary, the numerical parameters set forth in the specification and attached claims are approximations that may vary depending upon the desired performance sought to be obtained by the present invention.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

1-44. (canceled)

45. A composition for reducing the aggregate content of a protein preparation comprising:
(a) a first substrate comprising a first surface-bound ligand possessing a metal affinity functionality, the metal affinity functionality being substantially devoid of a metal, and
(b) a second surface-bound ligand different from the first surface-bound ligand, the second surface-bound ligand having an aggregate charge not opposite to that of the metal affinity functionality, wherein optionally a second substrate comprises the second surface-bound ligand.

46. The composition of claim 45, wherein the first substrate comprises the second-surface-bound ligand.

47. The composition of claim 45, wherein the first surface-bound ligand, the second surface- bound ligand, or both comprise a multidentate metal chelating moiety.

48. The composition of claim 45, wherein the first surface-bound ligand has an aggregate charge which is electronegative and is selected from the group consisting of iminodiacetic acid (2-(carboxymethylamino)acetic acid), ethylene glycol(aminoethylether)diacetic acid, nitriloacetic acid (2,2′,2″-nitrilotriacetic acid), aspartic acid, and glutamic acid.

49. The composition of claim 45, wherein the first surface-bound ligand has an aggregate charge which is electropositive and is selected from the group consisting of tris(2-aminoethyl)amine, diethylenetriamine, triethylenetetramine, tetraethylenepentamine, polypropylenimine tetraamine, and desferrioxamine.

50. The composition of claim 45, wherein at least one of the first substrate or the optional second substrate comprises a third surface-bound ligand which enhances the capacity of the composition to participate in hydrogen bonding, hydrophobic interactions, or pi-pi binding with a component of the protein preparation.

51. The composition of claim 45, wherein the first substrate, the optional second substrate, or both comprise more than one negatively charged metal chelating group in combination with a neutral group, a negatively charged group, or combinations thereof.

52. The composition of claim 45, wherein the first substrate, and the second substrate, or both comprise more than one positively charged metal chelating group in combination with a neutral group, a positively charged group, or combinations thereof.

53. The composition of claim 45, wherein the first surface-bound ligand and the second surface-bound ligand are covalently attached to the first or the second substrate.

54. The composition of claim 45, wherein the first substrate and the optional second substrate comprise a) a plurality of non-porous particles, or b) a plurality of porous particles comprising a pore size smaller than about 10 nm, between about 10 nm and 100 nm, or larger than about 100 nm.

55. The composition of claim 45, wherein the first substrate and the optional second substrate comprise a membrane, a monolith, a fiber, or a hollow fiber.

56. The composition of claim 55, wherein hollow fiber comprises a solid or porous walled hollow fiber.

57. The composition of claim 55, wherein the fiber comprises a permeable chopped fiber mat, an ordered wound, woven, or non-woven fiber structure, or a combination of a permeable chopped fiber mat and an ordered wound, woven, or non-woven fiber structure.

58. An apparatus comprising the composition of claim 1, wherein the apparatus is configured for chromatography.

59. The apparatus of claim 58, wherein the apparatus comprises a chromatographic device packed with the first substrate, the optional second substrate, or both.

60. The apparatus of claim 58, comprising one or more components comprising a chemical surface that is substantially inert or of sufficiently low surface area as to make no significant contribution to the chemical functionality of the apparatus and wherein the chemical surface of the component is configured to allow one or more of (1) create structural integrity, (2) direct flow of liquids therethrough, and (3) physically block, entrap, or entrain insoluble materials in the protein preparation to prevent them from interfering with the effective use of the device.

61. The apparatus of claim 58, wherein the first substrate or the optional second substrate comprise a) porous or non-porous particles, b) a porous membrane, c) non-porous fibers, d) porous reticular fibers, wherein the porous reticular fibers are hollow porous-walled fibers, e) fibrous filters, f) woven or crystalline frits, g) a monolith or a combination thereof.

62. The apparatus of claim 61, wherein the first substrate comprises the porous or non-porous particles and the second substrate comprises a) the porous membrane, b) the monolith or c) the woven or crystalline frits.

63. The apparatus of claim 61, wherein the porous or non-porous particles are a) sandwiched between the porous membrane or the monolith, b) sandwiched between the woven or amorphous fibrous filter, c) sandwiched between the woven or crystalline frits, d) embedded within a fibrous matrix, or d) embedded in a reticular polymer network.

64. The apparatus of claim 58, wherein the first surface-bound ligand, second surface-bound ligand, or both comprises a hydrogelatinous material.
